(19) United States
(12) Patent Application Publication
(10) Pub. No.: US 2026/0231999 A1
(43) Pub. Date: Aug. 13, 2026

(54) Thickened formula for allergic infants

(71) Applicant: N.V. Nutricia, Zoetermeer (NL)

(72) Inventors: Evan Abrahamse, Utrecht (NL); Lauriane Emmanuelle Mélanie Schwebel, Utrecht (NL); Sebastian Tims, Utrecht (NL); Leonardo Cornacchia, Utrecht (NL)

(73) Assignee: N.V. Nutricia, Zoetermeer (NL)

(21) Appl. No.: 19/177,669

(22) Filed: Apr. 14, 2025

Related U.S. Application Data
(62) Division of application No. PCT/EP2023/079700, filed on Oct. 25, 2023.

(30) Foreign Application Priority Data
Oct. 25, 2022 (EP) 22203651.9

(51) Int. Cl. A23L 33/00; A23C 9/16; A23L 33/175; A23L 33/18; A23L 33/20

(52) U.S. Cl. CPC A23L 33/40; A23C 9/16; A23L 33/175; A23L 33/18; A23L 33/20

(57) ABSTRACT

The invention concerns formula for based on extensively hydrolysed protein and/or free amino acids for allergic infants that are thickened with a combination of xanthan gum and locust bean gum.

### FIELD OF THE INVENTION

The field of the invention is formula for allergic infants.

### BACKGROUND OF THE INVENTION

Breast milk is the preferred method for infant feeding. When breastfeeding is not possible or the mother chooses not to breastfeed, infant formula are commercially available that are suitable as a complete nutrition. Usually these formulas are based on intact cow's milk protein, in particular whey protein and casein.

The prevalence of food allergy has been increasing in recent decades, particularly in developed countries. In early life, cow's milk allergy (CMA) is the most dominant food allergy with currently 2-5% of infants suffering. CMA in infancy represents an increasing global health and economic burden, which is caused not only by an increased prevalence over the last decades, but also by an increased persistence, severity and complexity of the condition. Besides acute clinical manifestations, which can be severe, CMA in early life can also have long-lasting effects, including delays in growth and development, as well as increased risk for the development of atopic diseases later in life. Hence, strategies to treat or prevent CMA are of major importance.

The standard dietary management of CMA in infants and children is allergen avoidance through elimination of cow's milk from the diet. A variety of formulas have been developed for the elimination of cow's milk protein. Extensively hydrolysed formulas (eHFs) are recommended for infants with mild CMA, whereas for infants with severe CMA and for infants that either do not tolerate eHFs or in which eHFs fail to resolve CMA symptoms, amino acid-based formulas (AAFs) are recommended.

However, a substantial amount of infants suffering from allergy, or at risk of becoming allergic, also suffer from functional gastro-intestinal disorders such as diarrhea and/or gastro-esophageal reflux (GER) including regurgitation. GER is the backward flow of stomach contents up the esophagus and sometimes even into or out of the mouth. Applying Rome IV criteria, regurgitation is the most prevalent functional gastro-intestinal disorder (FGID). The daily prevalence of regurgitation is 80% in babies up to 2 months, with 20% suffering from more than four regurgitations a day (Czinn et al, 2013, Pediatric Drugs 15:19-27). There is an association between cow's milk protein intolerance and gastro-esophageal reflux and these disorders often co-exist. Babies with CMA, representing 2-5% of the babies in Europe, are known to experience severe reflux. CMA causes sever gastric dysrhythmia and delayed gastric emptying, known to increase the severity of the regurgitations (Ravelli et al, 2001, JPGN, 59-64). With reflux the stomach content including hydrochloric acid, comes into contact with the esophagus, throat, nasal cavities, lungs and/or teeth, causing pain and damage. Overtime, repeated exposure of these areas with acid can cause increasing damage and cause more serious complications. Besides the problems already mentioned above, it may result in dehydration, impaired growth and/or a failure to thrive of the infant.

Anti-regurgitation formulas are available on the market, that have thickeners to increase the viscosity of the formula in the bottle and stomach. The thickeners typically used are starch and undigestible polysaccharides such as locust bean gum. Such formulas typically contain intact protein, such as whey protein and casein from cow's milk, and are therefore not suitable for allergic infants. An example is Nutrilon A.R. that has intact whey protein and casein from cow's milk and locust bean gum as a thickener. Therefore, there is a need of a formula suitable for infants suffering from food allergy that concomitantly has an anti-reflux or anti-regurgitation effect, in order to address both issues in one formula.

An infant formula available on the market and tailored to hypoallergenic formula is Allernova AR+ from Novalac containing a casein hydrolysate. The thickener mixture is made of locust bean gum, a mixture of two pectins and starch.

WO 2012/080462 discloses an anti-regurgitation formula containing at least one weakly esterified pectin, at least one strongly esterified pectin, and a thickener or gelling agent. Optionally the protein is hydrolysed.

WO 2004/60080 discloses extensively hydrolyzed, liquid nutritional formulas. Optionally thickeners can be present.

WO 2001/56406 discloses pediatric formulas comprising xanthan gum as a tolerance improver. Optionally the formula contains free amino acids or hydrolysed protein.

### SUMMARY OF THE INVENTION

The inventors set out to solve the problem of a suitable thickener or combination of thickeners that provided a proper viscosity in an infant formula suitable for allergic infants, that contains extensively hydrolysed whey protein and/or free amino acids as protein source. Aim was to ensure an anti-regurgitation effect. Criteria were that in the bottle the viscosity should be increased compared to a formula without a thickener, but not so thick that it is too difficult for an infant to suck through the hole of a teat. The thickeners should be efficient at a low concentration, as this will advantageously allow the presence of additional fibers such non-digestible oligosaccharide that improve the microbiota.

The thickeners should be suitable to be introduced into the powdered formula by simple dry blending and subsequent reconstitution should result in a stable product that is smooth and that does not have glossy transparent particles.

Upon testing many thickeners and their combinations and applying the above criteria, a combination of xanthan gum (XG) and locust bean gum (LBG) was found to be superior. This combination was subsequently tested in different concentrations and ratios under conditions that mimic the gastric digestion of an infant, and compared with a commercial anti-regurgitation product with intact protein that has clinically proven anti-regurgitation properties. Using these data, a mathematical model was developed to find the conditions where the extent and kinetics of viscosity were most similar. It was found that two very specific combinations of XG and LBG were the most effective in mimicking the viscosity under stomach conditions of the commercial AR product with intact proteins. It was found that one optimum of the weight ratio of XG to LBG lies between 0.8 and 1.2 and that another optimum of the weight ratio of XG to LBG lies between 1.5 and 6.

The XG/LBG combinations require considerably less thickener to be present in the formulas than typically found in the market of intact AR formulas, yet this composition unexpectedly gives advantageous similar high viscosity during gastric digestion and the best product properties.

Finally, testing XG and LBG in combination with non-digestible oligosaccharide in an in-vitro fermentation model using infant's microbiota showed a surprisingly improved effect on the acid metabolites production by the microbiota upon fermenting the combination, when compared to XG, LBG or the non-digestible oligosaccharide alone, and this effect was more similar to what is normally observed with the metabolites produced by the microbiota of exclusively breastfed infants.

### DETAILED DESCRIPTION OF THE INVENTION

The present invention thus concerns a nutritional composition comprising digestible carbohydrates, lipids, protein and thickener suitable for providing nutrition to an infant or young child suffering from cow's milk protein allergy, wherein the protein is or essentially consists of extensively hydrolysed protein and/or free amino acids and wherein the thickener comprises a combination of xanthan gum and locust bean gum. The present nutritional composition may in the form of a ready to drink liquid or in the form of a powder that upon reconstitution with water is a ready to drink liquid.

The present invention further concerns a nutritional composition comprising digestible carbohydrates, lipids, protein and thickener, wherein the protein is or essentially consists of extensively hydrolysed protein and/or free amino acids and wherein the thickener comprises a combination of xanthan gum and locust bean gum, for use in the dietary management of an infant or young child that suffers from allergy, preferably an infant or young child that suffers from cow's milk protein allergy.

This can also be worded as a method for the dietary management of an infant or young child that suffers from allergy, preferably an infant or young child that suffers from cow's milk protein allergy comprising administering to the infant a nutritional composition comprising digestible carbohydrates, lipids, protein and thickener, wherein the protein is or essentially consists of extensively hydrolysed protein and/or free amino acids and wherein the thickener comprises a combination of xanthan gum and locust bean gum. The invention can also be worded as use of lipids, digestible carbohydrates, protein, thickener and non-digestible oligosaccharide in the manufacture of a nutritional composition for the dietary management of an infant or young child that suffers from allergy, preferably an infant or young child that suffers from cow's milk protein allergy, wherein the protein is or essentially consists of extensively hydrolysed protein and/or free amino acids and wherein the thickener comprises a combination of xanthan gum and locust bean gum.

The present invention further concerns a nutritional composition comprising digestible carbohydrates, lipids, protein and thickener, wherein the protein is or essentially consists of extensively hydrolysed protein and/or free amino acids and wherein the thickener comprises a combination of xanthan gum and locust bean gum, for use in the treatment or prevention of regurgitation or gastro-esophageal reflux (GER) in an infant or young child that suffers from allergy, preferably in an infant or young child that suffers from cow's milk protein allergy.

This can also be worded as a method the treatment or prevention of regurgitation or gastro-esophageal reflux (GER) in an infant or young child that suffers from allergy, preferably in an infant or young child that suffers from cow's milk protein allergy, comprising administering to the infant a nutritional composition comprising digestible carbohydrates, lipids, protein and thickener, wherein the protein is or essentially consists of extensively hydrolysed protein and/or free amino acids and wherein the thickener comprises a combination of xanthan gum and locust bean gum.

The invention can also be worded as use of lipids, digestible carbohydrates, protein, thickener and non-digestible oligosaccharide in the manufacture of a nutritional composition for the treatment or prevention of regurgitation or gastro-esophageal reflux (GER) in an infant or young child that suffers from allergy, preferably in an infant or young child that suffers from cow's milk protein allergy, wherein the protein is or essentially consists of extensively hydrolysed protein and/or free amino acids and wherein the thickener comprises a combination of xanthan gum and locust bean gum.

Furthermore, the present invention concerns use of a combination of xanthan gum and locust bean gum as thickeners in a hypoallergenic nutritional composition that is suitable for an infant or young child that suffers from cow's milk protein allergy, wherein the hypoallergenic nutritional composition comprises digestible carbohydrates, lipids and protein, wherein the protein is or essentially consists of extensively hydrolysed protein and/or free amino acids.

The invention also concerns a process for preparing a nutritional composition comprising digestible carbohydrates, lipids, protein and thickener, wherein the protein is or essentially consists of extensively hydrolysed protein and/or free amino acids and wherein the thickener comprises a combination of xanthan gum and locust bean gum, the process comprising
a. providing a liquid nutritional composition comprising extensively hydrolysed protein and/or free amino acids as protein source, digestible carbohydrates, and lipids and optionally non-digestible oligosaccharide,
b. drying the liquid obtained in step a) to powder, preferably by spray drying, and
c. dry blending xanthan gum and locust bean gum and optional waxy starch, with the powder obtained in step b.

### Thickeners

The present invention is about a combination of xanthan gum and locust bean gum as thickeners for a formula that contains extensively hydrolysed whey protein and/or free amino acids as protein source.

The inventors have discovered that xanthan gum is a thickener which is suitable for use in a composition according to the invention, in particular in an infant formula, in combination with locust bean gum. Optionally also waxy starch is included in the thickener combination that is found particularly suitable for use in a composition according to the invention. The inventors have in particular demonstrated that such a combination provided excellent viscosity to a formula based on extensively hydrolysed protein or amino acids for allergic infants so as to reduce and prevent reflux or regurgitation in these infants. Combining XG and LBG allowed it them to be applied at an advantageously low concentration that still provided the desired increased viscosity yet further allowing the presence of having additional dietary fibers present to ensure desired prebiotic effects.

A lower concentration of thickeners in formula intended for allergic infants has the additional advantage that the chance of introducing contaminating, potentially allergenic-intact proteins together with the thickeners is reduced.

This selection of thickeners was made after an extensive testing of thickeners, its combinations and at different levels and concentrations, taking into account user experience, regulatory requirements, viscosity properties in the bottle and viscosity properties under infant stomach digestion conditions. It was found that xanthan gum as sole thickener at a level close to the allowed maximum did not provide sufficient viscosity in the bottle. Locust bean gum as sole thickener in formulas without intact protein does not have sufficient high viscosity under stomach conditions.

### Xanthan Gum

Xanthan gum, in the context of the present invention also indicated as xanthan, is a branched polysaccharide which is used as a food additive with the code E415. It is produced by the bacterium Xanthomonas campestris. It consists of a combination of four subunits: glucose, mannose, glucuronic acid and pyruvic acid. Xanthan gum is commercially available, for example Grinsted Xanthan gum from Dupont Danisco.

Preferably the total amount of xanthan gum is from 0.02 to 0.12 g/100 ml ready to drink liquid nutritional composition, more preferably from 0.03 to 0.09 g/100 ml, more preferably from 0.03 to 0.07 g/100 ml, more preferably from 0.04 to 0.05 g/100 ml ready to drink liquid nutritional composition. Preferably the total amount of xanthan gum is between 0.03 and 0.18 g/100 kcal, more preferably from 0.04 to 0.13 g/100 kcal, more preferably from 0.04 to 0.10 g/100 kcal, more preferably from 0.06 to 0.07 g/100 kcal. Preferably the total amount of xanthan gum is from 0.14 to 0.86 g per 100 g dry weight of the nutritional composition, more preferably from 0.21 to 0.64 g/100 g dry weight, more preferably from 0.21 to 0.50 g/100 g dry weight, more preferably from 0.29 to 0.36 g/100 g dry weight of the nutritional composition. If too much xanthan is present the gastric viscosity will be too high. If the amount is too low, a reduced effect on GER can be anticipated.

### Locust Bean Gum

Locust bean gum, in the context of the present invention also indicated as locust gum, carob gum, carob bean gum, carobin, is a galactomannan that is used as a food additive with the code E410. Locust bean gum can be isolated from seeds of the carob tree. It is composed of a straight backbone chain of D-mannopyranose units with a side-branching unit of D-galactopyranose having an average of one D-galactopyranose unit branch on every fourth D-mannopyranose unit.

Preferably the locust bean gum is cold soluble. Cold-soluble locust bean gum has a solubility in an aqueous medium at a temperature in the range from 10° C. to 45° C. of more than 60%. The cold-soluble locust bean gum is distinguished from native locust bean gum, in that it has a lower average molecular weight. Locust bean gum is commercially available, for example Grindsted LBG or LBG Cold soluble from Danisco-DuPont.

Preferably the total amount of locust bean gum is from 0.02 to 0.45 g/100 ml ready to drink liquid nutritional composition, more preferably from 0.03 to 0.30 g/100 ml. Preferably the total amount of locust bean gum is from 0.03 to 0.67 g/100 kcal, more preferably from 0.04 to 0.45 g/100 kcal. Preferably the total amount of locust bean gum is from 0.14 to 3.21 g per 100 g dry weight of the nutritional composition, more preferably from 0.21 to 2.14 g dry weight of the nutritional composition. If too much locust bean gum is present, the viscosity will be too high in the bottle. If the amount is too low, a reduced effect on GER can be anticipated.

### Waxy Starch

The compositions according to the invention may also comprise waxy starch as a further thickener, in particular precooked or pregelatinized waxy starch, preferably pregelatinized waxy starch. Waxy starch is derived for example from corn or potato or rice varieties that produce starch that typically contains at least 98% amylopectin. Therefore, as mentioned above, in the context of the present invention waxy starch is also defined as amylopectin. Waxy starch may also be denoted as glutinous starch or isoamylose. Dissolved waxy starch, or amylopectin, has a lower tendency of retrograding during storage and cooling, and is suitable as thickening agent. Starch is a mixture of 2 homopolymers, amylose and amylopectin, composed of D-anhydroglucopyranose that are linked to one another via α-(1-4) linkages and α-(1-6) linkages which are responsible for branches in the structure of the molecule. Amylose is slightly branched with short branches and the molecular weight of which can be between 10,000 and 1,000,000 Daltons. The molecule is made up of from 600 to 1000 glucose molecules; amylopectin or isoamylose, is a branched molecule with long branches every 24 to 30 glucose units by means of α-(1-6) linkages. Waxy starch or amylopectin is commercially available, for example waxy maize starch from Cargill. Waxy starch has a thickening effect in the bottle. Under stomach conditions, having been exposed to alpha-amylase upon digestion, waxy starch does not impose a high viscosity.

### Combinations of Xanthan Gum and Locust Bean Gum

It was found that a combination of XG and LBG was superior to XG or LBG alone. Preferably the combination of XG and LBG is present in the nutritional composition at a level of 0.05 to 0.50 g/100 ml, preferably 0.06 to 0.40 g/100 ml, even more preferably from 0.07 to 0.30 g/100 ml. Preferably the combination of XG and LBG is present in the nutritional composition at a level of 0.07 to 0.75 g/100 kcal, preferably 0.09 to 0.60 g/100 kcal, even more preferably from 0.10 to 0.45 g/100 kcal. Preferably the combination of XG and LBG is present in the nutritional composition at a level of 0.36 to 3.57 g/100 g dry weight, preferably 0.43 to 2.86 g/100 g dry weight, even more preferably from 0.50 g/100 g to 2.14 g/100 g dry weight. Preferably the wt/wt ratio of XG and LBG is from 0.1 to 6, preferably 0.5 to 5.

It was found that there are two optimum concentrations and ratios of the combination of XG and LBG. The viscosity under conditions mimicking digestion in the infant were most similar to that of the benchmark formula of Nutrilon A.R. with LBG and intact milk protein.

In a preferred embodiment the nutritional composition comprises a low amount of XG and LBG in a particular ratio. Preferably xanthan gum is present in an amount of 0.03 to 0.07 g/100 ml, preferably 0.03 to 0.06 g/100 ml, even more preferably 0.04 to 0.05 g/100 ml ready to drink nutritional composition. Preferably locust bean gum is present in an amount of 0.03 to 0.07 g/100 ml, more preferably 0.03 to 0.06 g/100 ml, even more preferably 0.04 to 0.05 g/100 ml ready to drink nutritional composition. Preferably the wt/wt ratio of xanthan gum to locust bean gum is from 0.8 to 1.2, more preferably 0.9 to 1.1. Preferably xanthan gum is present in an amount of 0.03 to 0.07 g/100 ml, preferably 0.03 to 0.06 g/100 ml, even more preferably 0.04 to 0.05 g/100 ml ready to drink nutritional composition and locust bean gum is present in an amount of 0.03 to 0.07 g/100 ml, more preferably 0.03 to 0.06 g/100 ml, even more preferably 0.04 to 0.05 g/100 ml ready to drink nutritional composition and the wt/wt ratio of xanthan gum to locust bean gum is from 0.8 to 1.2, more preferably 0.9 to 1.1.

Alternatively, according to this embodiment, xanthan gum is present in an amount of 0.04 to 0.10 g/100 kcal preferably 0.04 to 0.09 g/100 kcal, even more preferably 0.06 to 0.07 g/100 kcal. Preferably locust bean gum is present in an amount of 0.04 to 0.10 g/100 kcal, more preferably 0.04 to 0.09 g per 100 kcal, even more preferably 0.06 to 0.07 g/100 kcal. Preferably the wt/wt ratio of xanthan gum to locust bean gum is from 0.8 to 1.2, more preferably 0.9 to 1.1. Preferably xanthan gum is present in an amount of 0.04 to 0.10 g/100 kcal preferably 0.04 to 0.09 g/100 kcal, even more preferably 0.06 to 0.07 g/100 kcal and locust bean gum is present in an amount of 0.04 to 0.10 g/100 kcal, more preferably 0.04 to 0.09 g per 100 kcal, even more preferably 0.06 to 0.07 g/100 kcal and the wt/wt ratio of xanthan gum to locust bean gum is from 0.8 to 1.2, more preferably 0.9 to 1.1.

Alternatively, according to this embodiment, xanthan gum is present in an amount of 0.21 to 0.50 g/100 g dry weight of the nutritional composition, preferably 0.21 to 0.43 g/100 g dry weight, even more preferably 0.29 to 0.36 g/100 g dry weight. Preferably locust bean gum is present in an amount of 0.21 to 0.50 g/100 g dry weight of the nutritional composition, more preferably 0.21 to 0.43 g/100 g dry weight, even more preferably 0.29 to 0.36 g/100 g dry weight of the nutritional composition. Preferably, the wt/wt ratio of xanthan gum to locust bean gum is from 0.8 to 1.2, more preferably 0.90 to 1.1. Preferably, xanthan gum is present in an amount of 0.21 to 0.50 g/100 g dry weight of the nutritional composition, preferably 0.21 to 0.43 g/100 dry weight, even more preferably 0.29 to 0.36 g/100 g dry weight and locust bean gum is present in an amount of 0.21 to 0.50 g/100 g dry weight of the nutritional composition, more preferably 0.21 to 0.43 g/100 g dry weight, even more preferably 0.29 to 0.36 g/100 g dry weight of the nutritional composition and the wt/wt ratio of xanthan gum to locust bean gum is from 0.8 to 1.2, more preferably 0.90 to 1.1.

Using the combination of XG and LBG in such a low concentration is already sufficient to enable a desired high viscosity in the stomach, under conditions of a lower pH and digestion. The ratio of these thickeners being approximately 1 appeared to have a synergistic effect on the thickening properties, or viscosity increasing properties, especially at low pH. Although the viscosity in the bottle was slightly lower than the benchmark formula Nutrilon A.R., the viscosity is much higher than the nutritional composition that was not thickened and comparable or even slightly higher than a commercial formula with a mixture of thickeners and extensively hydrolysed milk protein (Allernova AR+).

For this embodiment a further improvement to increase the viscosity in the bottle to levels comparable to Nutrilon A.R. would be the further addition of waxy starch, also known as amylopectin. It was found that the presence of waxy starch results in an increased viscosity in the bottle, but not under stomach conditions. Preferably the total amount of waxy starch is from 0.4 to 1.5 g/100 ml ready to drink nutritional composition, more preferably from 0.4 to 0.8 g/100 ml. If too much waxy starch is present the viscosity will be too high, and it replaces more desirable sources of carbohydrates such as lactose. Starch is not desired as a digestible carbohydrate source in infant formula as it has a high glycemic index. If the amount is too low, the desired further improvement to increase the viscosity in the bottle will not be reached. In one embodiment, preferably xanthan is present in an amount of 0.03 to 0.07 g/100 ml ready to drink nutritional composition, more preferably 0.03 to 0.06 g/100 ml, even more preferably from 0.04 to 0.05 g/100 ml ready to drink nutritional composition, and locust bean gum is present in an amount of 0.03 to 0.07 g/100 ml ready to drink nutritional composition, preferably 0.03 to 0.06 g/100 ml, even more preferably from 0.04 to 0.05 g/100 ml ready to drink nutritional composition, and waxy starch is present in levels of 0.4 to 1.5 g/100 ml ready to drink nutritional composition, more preferably from 0.4 to 0.8 g/100 ml ready to drink nutritional composition and the wt/wt ratio of xanthan gum to locust bean gum is from 0.8 to 1.2, more preferably 0.9 to 1.1.

Alternatively, according to this embodiment, xanthan gum is present in an amount of 0.04 to 0.10 g/100 kcal preferably 0.04 to 0.09 g/100 kcal, even more preferably 0.06 to 0.07 g/100 kcal and locust bean gum is present in an amount of 0.04 to 0.10 g/100 kcal, more preferably 0.04 to 0.09 g per 100 kcal, even more preferably 0.06 to 0.07 g/100 kcal and waxy starch is present in levels of 0.6 to 2.2 g/100 kcal, more preferably from 0.6 to 1.2 g/100 kcal and the wt/wt ratio of xanthan gum to locust bean gum is from 0.8 to 1.2, more preferably 0.9 to 1.1

Alternatively, according to this embodiment, xanthan gum is present in an amount of 0.21 to 0.50 g/100 g dry weight of the nutritional composition, preferably 0.21 to 0.43 g/100 dry weight, even more preferably 0.29 to 0.36 g/100 g dry weight and locust bean gum is present in an amount of 0.21 to 0.10 g/100 g dry weight of the nutritional composition, more preferably 0.21 to 0.43 g/100 g dry weight, even more preferably 0.29 to 0.36 g/100 g dry weight of the nutritional composition and waxy starch is present in levels of 2.9 to 10.7 g/100 g dry weight of the nutritional composition, more preferably from 2.9 to 5.7 g/100 g dry weight of the nutritional composition and the wt/wt ratio of xanthan gum to locust bean gum is from 0.8 to 1.2, more preferably 0.90 to 1.1

In a different embodiment the nutritional composition comprises a low amount of XG and high amount LBG resulting in a particular ratio. Preferably, the sum of xanthan gum and locust bean gum is present in an amount of 0.13 to 0.40 g/100 ml ready to drink nutritional composition, preferably 0.20 to 0.40 g/100 ml. Preferably xanthan gum is present in an amount of 0.03 to 0.07 g/100 ml ready to drink nutritional composition, more preferably 0.03 to 0.06 g/100 ml. Preferably locust bean gum is present in an amount of 0.10 to 0.35 g/100 ml ready to drink nutritional composition, more preferably 0.20 to 0.30 g/100 ml. Preferably the wt/wt ratio of xanthan gum to locust bean gum is from 1.5 to 6.0, more preferably from 3.0 to 5.0. Preferably xanthan gum is present in an amount of 0.03 to 0.07 g/100 ml ready to drink nutritional composition, more preferably 0.03 to 0.06 g/100 ml and locust bean gum is present in levels of 0.10 to 0.35 g/100 ml ready to drink nutritional composition, more preferably 0.20 to 0.30 g/100 ml, and the wt/wt ratio of xanthan gum to locust bean gum is from 1.5 to 6.0, more preferably from 3.0 to 5.0.

Alternatively, according to this embodiment, the sum of xanthan gum and locust bean gum is present in an amount of 0.19 to 0.60 g/100 kcal, preferably 0.30 to 0.60 g/100 kcal. Preferably xanthan gum is present in an amount of 0.04 to 0.10 g/100 kcal, preferably 0.04 to 0.09 g/100 kcal. Preferably locust bean gum is present in an amount of 0.15 to 0.52 g/100 kcal, more preferably 0.30 to 0.45 g per 100 kcal. Preferably the wt/wt ratio of xanthan gum to locust bean gum is from 1.5 to 6.0, more preferably 3.0 to 5.0. Preferably xanthan gum is present in an amount of 0.04 to 0.10 g/100 kcal preferably 0.04 to 0.09 g/100 kcal, and locust bean gum is present in an amount of 0.15 to 0.52 g/100 kcal, more preferably 0.30 to 0.45 g per 100 kcal, and the wt/wt ratio of xanthan gum to locust bean gum is from 1.5 to 6.0, more preferably 3.0 to 5.0.

Alternatively, according to this embodiment, the sum of xanthan gum and locust bean gum is present in an amount of 0.93 to 2.86 g/100 g dry weight of the nutritional composition, preferably 1.43 to 2.86 g/100 g dry weight. Preferably xanthan gum is present in an amount of 0.21 to 0.50 g/100 g dry weight of the nutritional composition, preferably 0.21 to 0.43 g/100 g dry weight. Preferably locust bean gum is present in an amount of 0.71 to 2.50 g/100 g dry weight of the nutritional composition, more preferably 1.43 to 2.14 g/100 g dry weight. Preferably, the wt/wt ratio of xanthan gum to locust bean gum is from 1.5 to 6.0, more preferably 3.0 to 5.0. Preferably, xanthan gum is present in an amount of 0.21 to 0.50 g/100 g dry weight of the nutritional composition, preferably 0.21 to 0.43 g/100 dry weight, and locust bean gum is present in an amount of 0.71 to 2.50 g/100 g dry weight of the nutritional composition, more preferably 1.43 to 2.14 g/100 g dry weight, and the wt/wt ratio of xanthan gum to locust bean gum is from 1.5 to 6.0, more preferably 3.0 to 5.0.

As in this embodiment the viscosity in the bottle is sufficiently high, so there is no need to add waxy starch as an additional thickener. Hence in this different embodiment the amount of waxy starch is low or waxy starch is not present. Preferably the amount of waxy starch is less than 0.2 g/100 ml ready to drink nutritional composition, more preferably less than 0.1 g/100 ml. Alternatively, the amount of waxy starch is less than 0.3 g/100 kcal, more preferably less than 0.15 g/100 kcal. Alternatively, the amount of waxy starch is less than 1.4 g/100 g dry weight of the nutritional composition, more preferably less than 0.7 g/100 g dry weight.

### Protein Components

The protein source in the nutritional composition of the present invention is suitable for use in infants that suffer from allergy, in particular food allergy, more in particular an allergy for dietary protein, even more in particular cow's milk protein allergy. Therefore, the protein, or protein source, is extensively hydrolysed protein, free amino acids or a combination thereof.

The present invention advantageously concerns a composition, and use thereof, wherein the protein source preferably provides 7 to 20% of the total calories of the composition, more preferably the protein source provides 8 to 17% of the total calories, even more preferably the protein source provides 9 to 15% of the total calories of the composition. The present invention advantageously concerns a composition, and use thereof, wherein the protein source preferably provides 1.0 g to 3.5 g protein per 100 ml, more preferably the protein source provides 1.2 to 3.0 g per 100 ml, even more preferably the protein source provides 1.4 to 2.5 g/100 ml ready to drink nutritional composition. The present invention advantageously concerns a composition, and use thereof, wherein the protein source preferably provides 1.5 g to 5.2 g protein per 100 kcal, more preferably the protein source provides 1.8 to 4.5 g per 100 kcal, even more preferably the protein source provides 2.1 to 3.7 g/100 kcal. Alternatively, in the composition, and use thereof, according to the present invention, the amount of the protein source is preferably between 10 and 20 wt % based on dry weight of the total composition, preferably between 11 and 18 wt %, and even more preferably between 12 and 16 wt % based on dry weight of the total composition. Preferably extensively hydrolysed protein, free amino acids or a combination thereof is the sole protein source.

Extensively hydrolysed protein can be derived from cow's milk protein such as whey protein or casein. Extensively hydrolysed protein typically has been processed using an ultrafiltration step after the hydrolysis with proteolytic enzymes in order to remove the potentially allergenic intact protein and large peptides. Preferably the extensively hydrolysed protein is extensively hydrolysed whey protein.

The extensively hydrolysed protein, preferably extensively hydrolysed whey protein, has a degree of hydrolysis (of the protein) from 16 to 50%, more preferably from 20 to 30%, even more preferably from 20 to 25%. The degree of hydrolysis is defined as the percentage of peptide bonds which have been broken down by enzymatic hydrolysis, with 100% being the total potential peptide bonds present. A too low degree of hydrolysis will deliver an undesirable high level of intact allergenic protein or allergenic peptides.

In contrast, partially hydrolysed protein typically has a degree of hydrolysis (of the protein) from 5 to 15% which makes it suitable to induce tolerance as to some extent allergenic parts of the protein remain intact, yet makes it unsuitable for an infant or young child already suffering from allergy.

The peptide size and molecular weight distribution can be determined by routine methods known to the skilled person such as HPLC or size exclusion chromatography (SEC), in particular high performance size exclusion chromatography. Saint-Sauveur et al. “Immunomodulating properties of a whey protein isolate, its enzymatic digest and peptide fractions” Int. Dairy Journal (2008) vol. 18 (3) pages 260-270 describes an example thereof. In short, the total surface area of the chromatograms is integrated and separated into mass ranges expressed as percentage of the total surface area. The mass ranges are calibrated using peptides/proteins with a known molecular mass.

In the context of the present invention, the extensively hydrolysed protein preferably comprises from 55 to 85% peptides with a molecular weight below 1000 Da, from 15 to 45% peptides with a molecular weight between 1000 and below 5000 Da and from 0 to 1% peptides or proteins with a molecular weight of 5000 Da and above, all based on total protein of the hydrolysate. In a preferred embodiment the extensively hydrolysed protein, preferably the extensively hydrolysed whey protein, comprises peptides with the following size distribution: 60-90% with a size <1 kDa, 10-40% peptides with a size of 1 to <5 kDa, 0-0.5% peptides with a size of 5 to <10 kDa, and 0-0.2% peptides with a size >10 kDa, based on the total protein of the hydrolysate.

In a preferred embodiment of the present invention the extensively hydrolysed protein, preferably the extensively hydrolysed whey protein, comprises less than 100 mg peptides with a size above 5 kDa per gram protein hydrolysate, preferably less than 50 mg per gram protein hydrolysate, more preferably less than 10 mg per gram protein hydrolysate. Preferably the extensively hydrolysed protein, preferably extensively hydrolysed whey protein, comprises less than 30 mg peptides with a size above 10 kDa per gram protein hydrolysate, preferably less than 10 mg per gram protein hydrolysate, most preferably less than 5 mg per gram protein hydrolysate.

Preferably the extensively hydrolysed protein, preferably extensively hydrolysed whey protein, comprises less than 0.8 microgram, more preferably less than 0.2 microgram allergenic betalactoglobulin per g protein. In the context of the invention, the expression ‘allergenic betalactoglobulin’ refers to intact or immunogenic betalactoglobulin and does not account for the extensively hydrolysed betalactoglobulin. Allergenic betalactoglobulin can be determined by methods as known in the art, such as ELISA.

For those infants who have such a severe allergy that extensively hydrolysed protein, such as extensively hydrolysed whey protein, as protein source is still an issue, or who suffer from multiple food allergies, advantageously the protein source consists essentially of free amino acids. Preferably, the protein source comprises all essential amino acids. The optimal amino acid profile for infant formulas is known in the art and is present in amino acid based infant formulas such as Neocate. A preferred embodiment of an amino acid composition is given in example 9.

Non-digestible oligosaccharide Preferably nutritional composition of the invention additionally comprises one or more non-digestible oligosaccharides (NDO). These NDO may help in improving gastrointestinal disorders, often observed in allergic infants, and have an anti-allergic effect by improving the microbiota and gut barrier. As the total amount of fibers allowed in infant formulas is limited, relatively low concentrations of thickeners advantageously will allow for NDO to be additionally present in substantial amounts. It was found that the combination of XG, LBG and NDO showed an advantageous and synergistic effect on the function of the intestinal microbiota of an infant.

Advantageously and most preferred, the non-digestible oligosaccharide is water-soluble (according to the method disclosed in L. Prosky et al, J. Assoc. Anal. Chem 71:1017-1023, 1988) and is preferably oligosaccharide with a degree of polymerisation (DP) of 2 to 200. The average DP of the non-digestible oligosaccharide is preferably below 200, more preferably below 100, even more preferably below 60, most preferably below 40.

The non-digestible oligosaccharide is not digested in the intestine by the action of digestive enzymes present in the human upper digestive tract (small intestine and stomach). For example, glucose, fructose, galactose, sucrose, lactose, maltose and the maltodextrins are considered digestible. The oligosaccharide raw materials may comprise monosaccharides such as glucose, fructose, fucose, galactose, rhamnose, xylose, glucuronic acid, GalNac etc., but these are not part of the oligosaccharide.

The non-digestible oligosaccharide is fermented by the human intestinal microbiota. The non-digestible oligosaccharide is preferably prebiotic. The non-digestible oligosaccharide is preferably selected from the group consisting of fructo-oligosaccharides, non-digestible dextrin, galacto-oligosaccharides, xylooligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic acid oligosaccharides, sialyloligosaccharides and fuco-oligosaccharides, and mixtures thereof, preferably fructo-oligosaccharides.

A suitable type of oligosaccharide is long-chain fructo-oligosaccharides (lcFOS) which has an average degree of polymerization above 10, typically in the range of 10-100, preferably 15-50, most preferably above 20. A preferred type of long-chain fructo-oligosaccharides is inulin, such as Raftilin HP.

Another suitable type of oligosaccharide is short-chain oligosaccharide which has an average degree of polymerization (DP) of less than 10, preferably at most 8, preferably in the range of 2-7 In one embodiment, the nutritional composition comprises galacto-oligosaccharides, preferably beta-galacto-oligosaccharides, preferably trans-galacto-oligosaccharides. The galacto-oligosaccharides preferably have an average degree of polymerisation in the range of 2-8, preferably 3-7, i.e. are short-chain oligosaccharides in the context of the invention. (Trans) galactooligosaccharides are for example available under the trade name VivinaLGOS (Friesland Campina Domo Ingredients, Netherlands), Bimuno (Clasado), Cup-oligo (Nissin Sugar) and Oligomate55 (Yakult). In one embodiment the nutritional composition comprises short-chain fructo-oligosaccharides. The short-chain fructo-oligosaccharides preferably have an average degree of polymerisation in the range of 2-8, preferably 3-7, i.e. are short-chain short-chain fructo-oligosaccharides in the context of the invention. scFOS may be inulin hydrolysate products having an average degree of polymerization within the aforementioned (sub-) ranges; such scFOS products are for instance commercially available as Raftilose P95 (Orafti) or with Cosucra. scFOS alternatively can be enzymatically synthesized from sucrose by a fructosyltransferase. The short-chain non-digestible oligosaccharide may also comprise a mixture of galacto-oligosaccharides and/or fructo-oligosaccharides (i.e. scGOS and/or scFOS). In case the formula is based on free amino acids, the preferred NDO are not milk derived. Preferably the non-digestible oligosaccharide in such a formula is fructo-oligosaccharides.

The nutritional composition may contain a mixture of two or more types of non-digestible oligosaccharides, most preferably a mixture of two non-digestible oligosaccharides. In case the NDO comprises or consists of a mixture of two distinct oligosaccharides, one oligosaccharide may be short-chain as defined above and one oligosaccharide may be long-chain as defined above. Most preferably, short-chain oligosaccharides and long-chain oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1. Suitable mixtures include mixtures of long-chain fructo-oligosaccharides with short-chain fructo-oligosaccharides or with short-chain galacto-oligosaccharides. Hence, in one embodiment, short-chain galacto-oligosaccharides and long-chain fructo-oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1. Also, in one embodiment, short-chain fructo-oligosaccharides and long-chain fructo-oligosaccharides are present in a weight ratio short-chain to long-chain in the range of 1:99-99:1, more preferably 1:1-99:1, more preferably 4:1-97:3, even more preferably 5:1-95:5, even more preferably 7:1-95:5, even more preferably 8:1-10:1, most preferably about 9:1.

In particular it was found that a combination of XG and LBG, long-chain fructo-oligosaccharides and short-chain oligosaccharides selected from galacto-oligosaccharides and fructo-oligosaccharides, showed an advantageous and synergistic effect on the function of the intestinal microbiota of an infant. Adding a mixture of short-chain galacto-oligosaccharides with long-chain fructo-oligosaccharides or a mixture of short-chain fructo-oligosaccharides with long-chain fructo-oligosaccharides to the combination of XG and LBG resulted in a reduced amount and reduced relative amounts of butyrate and propionate and gas, and this reduction was higher than expected based on the single thickeners or non-digestible oligosaccharides. In the presence of short-chain galacto-oligosaccharides furthermore the amount of lactic acid was synergistically increased. The combination of these specific thickeners and specific non-digestible oligosaccharides therefore synergistically shifts the activity of infant intestinal microbiota in a direction that is more similar to that observed in exclusively breastfed infants. Thus in one embodiment the present nutritional composition comprises non-digestible oligosaccharide which is a mixture of long-chain fructo-oligosaccharides and short-chain-fructo-oligosaccharides or is a mixture of long-chain fructo-oligosaccharides and short-chain galacto-oligosaccharides.

The nutritional composition preferably comprises 0.05 to 20 wt % of said non-digestible oligosaccharide, more preferably 0.5 to 15 wt %, even more preferably 1 to 10 wt %, most preferably 2 to 10 wt %, based on dry weight of the nutritional composition. When in liquid form, the nutritional composition preferably comprises 0.01 to 1.0 g non-digestible oligosaccharide, more preferably 0.25 to 0.8 g, even more preferably comprises at least 0.4 g/100 ml ready to drink nutritional composition, preferably from 0.4 to 0.8 g, more preferably from 0.6 to 0.8 g/100 ml ready to drink nutritional composition.

### Probiotics

Preferably the nutritional composition comprises lactic acid producing bacteria selected from the group consisting of the genera Bifidobacterium and/or Lactobacillus, in particular Bifidobacterium. The intestinal microbiota of breastfed infants is high in bifidobacteria. Addition to the nutritional composition of one or more strains belonging to the Bifidobacterium genus will further improve the intestinal microbiota and its activity. More preferably the nutritional composition comprises Bifidobacterium breve. Such strains of the species of Bifidobacterium breve are commercially available or can be isolated from the microbiota of infants. An example of a commercially available strain is Bifidobacterium breve M-16V from Morinaga. The amount of Bifidobacterium and/or Lactobacillus is preferably 104 to 1011 cfu per gram dry weigh of the nutritional composition.

### Nutritional Composition

The nutritional composition according to the invention can be used as an infant or follow-on formula, as a nutritional therapy, as a food for special medical purposes or as a nutritional supplement. The nutritional composition is preferably an oral composition. The nutritional composition is administered orally to, or intended to be administered orally to, a subject in need thereof, in particular to children and infants, including toddlers, preferably children up to 6 years of age, preferably infants or young children typically with an age of 0-36 months, more preferably infants 0-12 months of age, most preferably 0-6 months of age.

Thus, in some embodiments, the nutritional composition is an infant formula, follow-on formula or young child formula (also referred to as growing-up milk or toddler milk), preferably it is an infant formula or follow-on formula, most preferably an infant formula. The terms ‘infant formula’ and ‘follow-on formula’ are well-defined and controlled internationally and consistently by regulatory bodies. It recommends for nutritional value and formula composition, which require the prepared milk to contain per 100 ml not less than 60 kcal (250 kJ) and no more than 70 kcal (295 kJ) of energy. The EU, FDA and other regulatory bodies have set nutrient requirements in accordance therewith. This caloric density ensures an optimal ratio between water and calorie consumption.

Suitably, the nutritional composition is in a powdered form, which preferably can be reconstituted with water to form a liquid. When the nutritional composition is in a liquid form, the preferred volume administered on a daily basis is in the range of about 80 to 2500 ml, more preferably about 450 to 1000 ml per day.

The nutritional composition according to the invention comprises lipids, preferably lipids suitable for infant nutrition as known in the art. The lipids of the nutritional composition preferably provide 2.8 to 7.0 g, more preferably 4.0 to 6.0 g per 100 kcal of the nutritional composition. When in liquid form, the nutritional composition preferably comprises 1.9 to 4.7 g lipids per 100 ml, more preferably 2.7 to 4.0 g per 100 ml. Based on dry weight the present nutritional composition preferably comprises 12.5 to 40 wt % lipids, more preferably 19 to 30 wt %.

The nutritional composition according to the invention may further comprise long-chain polyunsaturated fatty acids (LC-PUFA). LC-PUFA are fatty acids wherein the acyl chain has a length of 20 to 24 carbon atoms and wherein the acyl chain comprises at least two unsaturated bonds between carbon atoms in the acyl chain. More preferably the nutritional composition comprises at least one LC-PUFA selected from the group consisting of eicosapentaenoic acid (EPA, 20:5 n3), docosahexaenoic acid (DHA, 22:6 n3), arachidonic acid (ARA, 20:4 n6) and docosapentaenoic acid (DPA, 22:5 n3), preferably the nutritional composition comprises at least DHA. Such LC-PUFA have a further beneficial effect on reducing the risk for allergy. The preferred content of LC-PUFA in the nutritional composition does not exceed 15 wt % based on total fatty acids, preferably does not exceed 10 wt %, even more preferably does not exceed 5 wt %. Preferably the nutritional composition comprises at least 0.2 wt %, preferably at least 0.25 wt %, more preferably at least 0.35 wt %, even more preferably at least 0.5 wt % LC-PUFA based on total fatty acids. The amount of DHA is preferably at least 0.2 wt %, more preferably at least 0.3 wt %, more preferably at least 0.35 wt %, even more preferably 0.35-0.6 wt % based on total fatty acids.

The nutritional composition comprises digestible carbohydrates. Typically, digestible carbohydrates that are known in the art to be suitable for use in infant nutritional compositions, for example selected from digestible polysaccharides (e.g. starch, maltodextrin), digestible monosaccharides (e.g. glucose, fructose), and digestible disaccharides (e.g. lactose, sucrose). Particularly suitable is lactose and/or maltodextrin. Preferably the nutritional composition comprises lactose. Lactose is the main digestible carbohydrate in milk and has a relatively low glycemic index. For those infants that need an amino acid based formula, lactose is not desired as it is derived from milk. In that case preferably the nutritional composition comprises maltodextrin. Maltodextrin consists of D-glucose units connected in chains of variable length. The glucose units are primarily linked with a (1→4) glycosidic bonds and is typically composed of a mixture of chains that vary from three to 17 glucose units long. Maltodextrins are classified by DE (dextrose equivalent) and in case maltodextrin is included in the present nutritional composition as digestible carbohydrate, the maltodextrin preferably has a DE from 3 to 47.

Minerals, vitamins, trace elements and other micronutrients are present as known in the art to comply with the directives on infant and follow-on formulas and food for medical purposes intended for infants.

As the nutritional composition according to the invention contains thickeners, the shear viscosity of the reconstituted ready to drink nutritional composition or formula, is higher than that of standard infant formulas without thickeners. The shear viscosity is measured at 37° C. at a shear rate of 10 s⁻¹ as measured by an Anton Paar viscometer. Preferably the ready to drink nutritional composition, preferably in the bottle, has a shear viscosity from 20 to 100 mPa·s, more preferably from 25 to 90 mPa·s, even more preferably from 30 to 80 mPa·s. These are viscosities as observed in thickened anti-regurgitation formulas that have a clinically proven effect on treating and preventing reflux.

### Application

The composition according to the present use is preferably enterally administered, more preferably orally. The present composition can advantageously be applied as a complete nutrition for infants.

The nutritional composition of the present invention is preferably suitable for use in providing nutrition to food allergic subjects. The present nutritional composition is specifically intended for use in food allergic infants and/or food allergic toddlers, more preferably infants. Infants have an age of 0-12 months, toddlers have an age of 12-36 months. Thus the food allergic subject is preferably a food allergic infant and/or toddler. The subjects have a food allergy, in particular an allergy for a dietary protein, more preferably suffer from cow's milk protein allergy. The nutritional composition of the present invention is preferably used to treat the food allergy, more preferably cow's milk protein allergy. The nutritional composition of the present invention is preferably used in the dietary management of food allergy, more preferably cow's milk protein allergy.

Preferably the present nutritional composition is for use the treatment and/or prevention, preferably the prevention of reflux, in subjects with a food allergy. Reflux, in context of present invention also referred to as gastro-esophageal reflux (GER), is the backward flow of stomach contents up the esophagus and sometimes even into or out of the mouth. At the lower end of the esophagus, the lower esophageal sphincter (LES) opens when food is swallowed and then normally closes again to keep stomach contents in place. When the LES is not working properly, with reflux the stomach content including hydrochloric acid, comes into contact with the esophagus, throat, nasal cavities, lungs and/or teeth. The diagnostic term regurgitation is used when the reflux can be seen. Regurgitation is the most prevalent functional gastro-intestinal disorder (FGID) and has been estimated to affect as many as 30% of the infants worldwide and 80% of infants up to 2 months of age. When the regurgitation of gastric contents causes complications or contributes to tissue damage or inflammation it is called gastro-esophageal reflux disease (GERD). Reflux and GERD, are problems especially during infancy. Billead et al 1990 EJCN 44, 577-583, show that infants with GER have a slightly more rapid gastric emptying. In infants (with or) without GER, gastric residual content is lowest in case of whey protein hydrolysate and human milk ingestion and higher when casein is ingested. Gastric emptying is also more rapid in whey protein dominant formula. Tolia: JPGN 1992, 15, 297-301 discloses that with whey protein hydrolysates, the gastric emptying rate is higher than with soy protein or casein containing formulas. Also Staelens 2008 Clin Nutr 27, 264-268 show that an extensively hydrolysed whey formula is better tolerated and the stomach is emptied faster. This means that it is especially important to mimic the viscosity under gastric digestion conditions at the first 50 minutes of digestion. After 70 minutes it is less relevant to have a high viscosity and the stomach already has emptied for the most part when formulas with extensively hydrolysed protein are ingested. On the contrary, a higher viscosity after 70 to 120 min is less desired as this implies that the nutrition may have a too high viscosity in the small intestine and may impair bio-accessibility of for example micronutrients.

The combination of thickeners of the nutritional composition of the present invention resulted in a viscosity in the bottle and viscosity kinetics of hypoallergenic formulas under gastric digestion conditions that were very similar to a commercial formula proven to have anti-regurgitation properties (Bellaiche et al, 2022, JPGN 73:579-585). It is therefore plausible that these hypoallergenic formulas with a combination of xanthan gum, locust bean gum, and optionally waxy starch, according to the present invention will treat and prevent reflux/regurgitation in subjects and also will treat and prevent reflux/regurgitation in subjects suffering from food allergy, in particular cow's milk protein allergy.

In one aspect the present invention provides an advantageous effect on the intestinal microbiota in an infant or young child. It was found that the combination of thickeners together with non-digestible oligosaccharide showed a surprisingly improved effect on the acid metabolites production by intestinal microbiota, when compared to xanthan gum or locust bean gum or the non-digestible oligosaccharide alone, and this effect was more similar to what is normally observed with the metabolites produced by the microbiota of exclusively breastfed infants. In particular using as non-digestible oligosaccharides a mixture of galacto-oligosaccharides with long-chain fructo-oligosaccharides or a mixture of short-chain fructo-oligosaccharides with long-chain fructo-oligosaccharides together with the combination of xanthan gum and locust bean gum, resulted in a reduced amount and reduced relative amounts of butyrate and propionate and gas, and this reduction was higher than expected based on the single thickeners or non-digestible oligosaccharides. Furthermore, in the presence of short-chain galacto-oligosaccharides, the amount of lactic acid was synergistically increased. Hence, the combination of these specific thickeners and specific non-digestible oligosaccharides therefore synergistically shifts the activity of infant intestinal microbiota in a direction that is more similar to that observed in exclusively breastfed infants.

Thus the present invention also concerns a nutritional composition according to the invention as defined herein, for use in modulating the intestinal microbiota activity in infants or young children, preferably infants, towards the activity as found in the microbiota of healthy breastfed infants, wherein the modulation is at least one, preferably at least two, selected from
a. increasing the amount of acetate or relative amount of acetate based on total SCFA,
b. increasing the amount of L-lactate,
c. decreasing the amount of gas production,
d. decreasing the amount of propionate or relative amount of propionate based on total SCFA, and
e. decreasing the amount of butyrate or relative amount of butyrate based on total SCFA,
   when compared to the intestinal microbiota activity in the same group of infants or young children, that are fed a nutritional composition that does not comprise a combination of both xanthan gum and locust bean gum thickeners and non-digestible oligosaccharide as defined in any one of the preceding claims.

In other words, the invention concerns a method for modulating the intestinal microbiota activity in infants or young children, preferably infants, the method comprising administering the nutritional composition according to the invention as defined herein, and the intestinal microbiota activity is modulated towards the activity as found in the microbiota of healthy breastfed infants, wherein the modulation is at least one, preferably at least two, selected from
a. increasing the amount of acetate or relative amount of acetate based on total SCFA,
b. increasing the amount of L-lactate,
c. decreasing the amount of gas production,
d. decreasing the amount of propionate or relative amount of propionate based on total SCFA, and
e. decreasing the amount of butyrate or relative amount of butyrate based on total SCFA,
   when compared to the intestinal microbiota activity in the same group of infants or young children, that are fed a nutritional composition that does not comprise a combination of both xanthan gum and locust bean gum thickeners and non-digestible oligosaccharide as defined in any one of the preceding claims.

Also, the present invention concerns a nutritional composition according to the invention as defined herein, for use in modulating the intestinal microbiota activity in infants or young children, preferably infants, towards the activity as found in the microbiota of healthy breastfed infants, wherein the intestinal microbiota activity is determined by one or more of
a. the amount of acetate or relative amount of acetate based on total SCFA,
b. the amount of L-lactate,
c. the amount of gas production,
d. the amount of propionate or relative amount of propionate based on total SCFA, and
e. the amount of butyrate or relative amount of butyrate based on total SCFA.

In other words, the invention concerns a method for modulating the intestinal microbiota activity in infants or young children, preferably infants, the method comprising administering the nutritional composition according to the invention as defined herein, and the intestinal microbiota activity is modulated towards the activity as found in the microbiota of healthy breastfed infants, wherein the intestinal microbiota activity is determined by one or more of
1. the amount of acetate or relative amount of acetate based on total SCFA,
2. the amount of L-lactate,
3. the amount of gas production,
4. the amount of propionate or relative amount of propionate based on total SCFA, and
5. the amount of butyrate or relative amount of butyrate based on total SCFA.

### EXAMPLES

### Example 1: Locust Bean Gum as Sole Thickener in Formulas Without Intact Protein does not have Sufficient High Viscosity Under Stomach Conditions

### Tested Formulas Were:

1) Pepti Syneo IF, a commercial infant formula comprising extensively hydrolysed whey protein and no thickener.
2) Nutrilon A.R.1, a commercial anti-regurgitation infant formula comprising intact protein (casein and whey protein) and as thickener 0.5 g LBG/100 ml. This formula with intact protein has been clinically proven to be efficacious against regurgitation and is the reference in term of viscosity values.
3) Pepti Syneo IF with 0.5 g LBG/100 ml. LBG (Grindsted LBG 860, Dansico 10120192) was dryblended into the powdered composition to yield the final concentration of 0.5 g/100 ml.
4) Pepti Syneo IF with 2 g/100 ml waxy maize starch (Waxy maize starch (Hiform, Cargill, Haubourdin, France).
5) Allernova AR+a commercially available anti-regurgitation infant formula comprising extensively hydrolysed casein, a mixture of highly and weakly esterified pectin, tapioca starch and locust bean gum as thickener.

The powdered formulas were reconstituted with 37° C. prewarmed tap water according to the instructions of the manufacturer and the bottle was shaken by hand for 30 seconds. The reconstituted samples were held in the water bath maintained at 37° C.

### Semi-Dynamic Stomach Model

Gastric digestion of the thickened formulas was simulated in bioreactors using a fermenter set-up (Dasgip, Eppendorf, Germany), allowing pH control, substrate pump and overhead agitation at 60 rpm in a 37° C. water bath. The in vitro digestion protocol was chosen according to the INFOGEST consensus (Minekus et al., 2014, Food Funct. 5, 1113-1114), with small modifications. Adaptations included the simulation of continuous swallowing of unstimulated saliva (Havenaar et al., 2013, Int J Parma 457:327-332; Bourlieu et al., 2014, Crit Rev Food Sci Nutr 54:1427-1457; Davis et al, 2009, Psychoneuroendocrinology, 34:795-804). The model was upscaled to 2:1 to mimic a meal intake of 200 mL of formula ingested by a 6 months infant.

After reconstitution, 400 mL of infant formula was placed in the bioreactor. Initial sampling started after 10 min, followed by 5 min of pH equilibration to 6.2. Then, a volume of 66 mL of simulated stimulated saliva fluid (106 mM NaCL, 30 mM KCl, 2.0 mM CaCl₂), 0.6 mg/mL α-amylase (SIGMA, A9857)) was introduced into the bioreactor. In the meantime continuous unstimulated saliva fluid (106 mM NaCl, 30 mM KCl, 2.0 mM CaCl₂), 0.38 mg/mL α-amylase (SIGMA, A9857)) was added, together with simulated gastric juice residue (106 mM NaCl, 30 mM KCl, 0.51 mM CaCl₂), 0.05 mg/mL pepsin (SIGMA P6887), 0.125 mg/mL lipase (SIGMA, 80612)) to represent the entrance of the formula in the stomach of the infant. The gastric digestion simulation period lasted 120 min with continuous addition of simulated unstimulated saliva (1.5 mL) and simulated gastric juice (140 mL), and acidification down to pH=4,4 (using HCl 0.5M). The bioreactor content was regularly sampled for rotational rheological viscosity measurement at 0, 10, 30, 50, 70, 90 and 120 min in simulated gastric digestion.

### Viscosity Measurements

The rheological behavior of each sampling was evaluated using a rheometer Anton Paar MCR101 with a plate-plate geometry (PP50) positioned at a gap of 1 mm and heated at 37° C. After a pre-shear of 10 sec at 5 s⁻¹, and another 10 sec waiting period, the measuring shear ramp from 2 to 100 s⁻¹. Viscosity values are given at a shear rate of 10 s⁻¹, typically representing the gastric shear and expressed in means+/−SEM of 3 to 8 repetitions.

The results are shown in Table 1. The extensively hydrolysed formula without LBG (1) showed a significantly lower viscosity compared to the anti-regurgitation formulas (2 and 5). The extensively hydrolysed formula supplemented with 0.5 g/100 ml LBG (3) achieved a similar viscosity level as the anti-regurgitation formulas (2 and 5) after reconstitution in the bottle. However, under stomach conditions after 30 min, a significantly lower viscosity level was observed. Thus, the solution of adding LBG does not work for eHF formulas. The interaction in the stomach between LBG and intact proteins, such as casein, causes a sufficient stomach viscosity, but because such proteins are lacking in the extensively hydrolysed anti-allergic formula the observed stomach viscosity is too low. Furthermore, the amount of LBG used (0.5 g/100 ml) is higher than desired as this leaves insufficient room in the recipe for a desired amount of prebiotics such as a scGOS/lcFOS mixture. Lower amounts of LBG however resulted in a too low viscosity in the bottle when compared to the standard anti-regurgitation formula (data not shown). Using starch as sole thickener (4) resulted in a good viscosity in the bottle, but under stomach conditions after 30 min the viscosity was similar as a formula without a thickener. A commercial AR formula with a mixture of thickeners (Tapioca starch, LBG, HMP and LMP; 5) showed a lower viscosity in the bottle, but viscosity was higher after 50 to 70 min. A too high viscosity at the end of the stomach digestion before it enters the duodenum is not desired as this may impact mineral bioavailability.

TABLE 1
Viscosities at 37° C. and at 10 s⁻¹ shear rate of infant
formulas under conditions of infants stomach digestion

| | Formula | | | | | | | | | |
|---|---|---|---|---|---|---|---|---|---|---|
| | 1) eHF formula | | 2) AR formula | | 3) eHF formula | | 4) eHF | | 5) AllernovaA+ | |
| | Thickener g/100 ml | | | | | | | | | |
| | | | | | | | | | Tapioca | |
| | | | | | | | | | starch, LBG, | |
| | — | | LBG 0.48 | | LBG 0.5 | | waxy starch 2 | | HMP and LMP | |
| Time [min] | Viscosity (s.d.) [mPa · s] | | | | | | | | | |
| 0 | 13.7 | (10.5) | 78.8 | (9.7) | 73.1 | (14.8) | 80.1 | (7.4) | 48.5 | (2.4) |
| 10 | 13.6 | (8.0) | 18.8 | (5.6) | 18.9 | (2.9) | 22.2 | (19.2) | 18.2 | (0.9) |
| 30 | 2.8 | (3.6) | 13.8 | (5.1) | 6.0 | (1.4) | 2.2 | (0.9) | 17.0 | (0.9) |
| 50 | 1.4 | (1.3) | 12.2 | (4.6) | 3.5 | (0.4) | 1.5 | (0.3) | 12.9 | (1.4) |
| 70 | 3.8 | (5.8) | 8.6 | (2.6) | 2.8 | (1.1) | 1.6 | (0.4) | 13.0 | (1.0) |
| 90 | 1.6 | (0.4) | 5.8 | (1.4) | 2.3 | (0.2) | 2.2 | (1.3) | 12.9 | (1.0) |
| 120 | 1.0 | (0.4) | 3.1 | (0.6) | 1.9 | (0.3) | 1.3 | (0.1) | 12.9 | (2.1) |

### Example 2: Selecting Combinations of Thickeners to Prepare Formulas with Extensively Hydrolysed Whey Protein to Have Adequate Viscosity and Product Technological Properties After Reconstitution to a Ready to Drink Formula

Several recipes for infant formulas with extensively hydrolysed protein were developed, taking into account that i) the maximum amount of total fiber, including non-digestible thickeners, should be 0.8 g/100 ml, ii) the prebiotic fiber combination such as GOS/lcFOS or scFOS/lcFOS should be present in an amount of at least 0.4 g/100 ml (and preferably higher), iii) the regulatory required amounts of protein, calories and calorie contribution of fat and protein and carbohydrates should be met, and iv) the addition of thickeners would not dilute the existing recipe too much in order to ensure that the requirements of the minimal amounts of DHA can be met, without the need to add extra DHA.

As a benchmark, the viscosity in the bottle and under stomach conditions should be comparable to that of Nutrilon A.R.1 (with 0.48 g LBG/100 ml). The viscosity should also not be too high, either in the bottle or under stomach conditions after 70 to 90 min of digestion, as this impacts drinking through a teat, and potentially may impact bioavailability of (micro) nutrients, respectively.—Other criteria were user experience and product stability related as explained in more detail.

### Thickeners Tested

Waxy maize starch (Hiform, Cargill, Haubourdin, France)
Xanthan gum (Keltrol Grindsted 808 MAS-SH clear form CP Kelco or IFF)
Locust bean gum (Grindsted LBG 860, Danisco, Valencia, Spain)
Beta-glucan, (PromOat, Lantmannen, Kimstad, Sweden)
Amidated low methylated pectin, (Amid CF 010-D, HERBSTREITH & FOX GmbH & Co, Neurenberg,
Non-amidated low methylated pectin (Classic CF 714, HERBSTREITH & FOX GmbH & Co, Neurenberg, Germany)
Amidated high methylated pectin (Amid CS 005, HERBSTREITH & FOX GmbH & Co, Neurenberg, Germany)
Non-amidated high methylated pectin (Classic CU 70, HERBSTREITH & FOX GmbH & Co, Neurenberg, Germany)

Over 40 single and combinations of thickeners with varying concentrations were tested. Different concentrations of each thickening agents were weighted, and then dry blended to the powdered hydrolysed infant formula, Pepti Syneo®. Reconstitution and viscosity measurements were performed as described in example 1.

The viscosity measurements were carried out with a rheometer (Anton Paar GmbH MCR302, Austria) equipped with a thermostatic bath at a temperature of 37° C. Viscosity values are given at a shear rate of 10 s⁻¹,

### Results:

### Single Fibers:

0.5 g/l LBG as sole thickener resulted in a satisfactory viscosity in the bottle, in accordance with example 1. The product was stable and no large “fish eyes” were observed. “Fish eyes” are transparent glossy particles and when too large this is not only visually unattractive for the consumer, it also may impact the passage of the formula through a teat. Lower concentrations of LBG resulted in viscosities in the bottle that were too low when compared to Nutrilon A.R.1. However, as is known from example 1, a too low viscosity under stomach conditions is observed when 0.5 g LBG/100 ml is used for formulas that have extensively hydrolysed protein or free amino acids.

It was found that waxy starch alone as a thickener was not suitable, as high amounts are needed to improve the bottle viscosity to a sufficiently high level (2 g/100 ml) and this is giving recipe constraints. The product was stable and no large “fish eyes” were observed. Concentrations of 1.5 g/100 ml waxy starch or lower resulted in a formula viscosity below that of Nutrilon A.R.1. Furthermore the viscosity under stomach conditions is not affected by waxy starch as the alpha-amylase in the saliva will degrade the waxy starch, resulting in a too low viscosity under stomach conditions as can be seen in example 1.

Using beta-glucan (0.4 g/100 ml) as a thickener in the extensively hydrolysed protein formula resulted in a highly unstable product that showed separation, that also had a too low viscosity in as far as this could be measured in the separated product.

The use of single pectin (0.3 g/100 ml) of either HM, HMA, LM or LMA pectin) resulted in formulas that had too low viscosity. These formulas were unstable, in that the product separated. Increasing pectin levels can be expected to further destabilize the formulas. In addition large “fish eyes” were observed. It is of relevance that the thickeners can be dry blended into the base powders of the infant formulas and not need to be processed in the wet phase of the manufacturing process, as this is for commercial production unattractive and gives as a low recipe flexibility, and may give technical problems when spray drying. Therefore these pectins were less attractive.

Xanthan gum as single thickener with a level close to the allowed maximum (0.1 g/100 ml) resulted in a formula with too low viscosity in the bottle compared to Nutrilon A.R.1.

### Combinations of Two Fibers:

A pectin mixture of HM(A) and LMA pectin (0.2 g/100 ml each) resulted in a formula that was too thin in the bottle compared to Nutrilon A.R.1. The product was not stable. As with the single fibers, large “fish eyes” were observed.

Mixtures of pectin with waxy starch only showed a sufficiently high viscosity in the bottle when 2 g/100 ml starch was used, which gives recipe constraints. Less waxy starch resulted in a too thin formula. In general many of the combinations tested wherein the thickeners were dry blended into the powdered formula were unstable and/or showed large “fish eyes”.

Pectin with XG mixtures with concentrations as maximally allowed by regulation where too thin when compared with Nutrilon A.R.1.

LBG with waxy starch showed a sufficient viscosity in the bottle, but as waxy starch does not contribute to the viscosity in the stomach the same low viscosity LBG in an extensively hydrolysed protein formula under stomach conditions can be expected.

Combinations of LBG with XG showed a promising viscosity level in the bottle, but this was concentration and ratio dependent. If the weight ratio was close to 1, i.e. between 0.7 and 1.5 the total amount of the two thickeners should be between 0.05-0.2 g/100 ml. Above 0.2 g/100 ml the formula was too thick. When the total amount of 2 thickeners was below 0.05 g/100 ml the viscosity was much lower than Nutrilon A.R.1.

For combinations of LBG and XG where the amount of locust bean gum was higher than XG, i.e. a ratio above 3, good viscosities were observed with LBG concentrations of at least 0.15 g/100 ml and at most 0.35 g/100 ml, while the amount of xanthan gum should at least be 0.03 g/100 ml and at most 0.07 g/100 ml.

### Combinations of 3 Fibers:

Combinations of XG, LBG and waxy starch showed good results on viscosity if the weight ratio of XG and LBG was close to 1, i.e. between 0.7 and 1.5 and the total amount of XG and LBG was not above 0.2 g/100 ml or below 0.05 g/100 ml. Especially in the range 0.5 to 1.5 g/100 ml XG+LBG, the additional presence of a relatively low amount of waxy starch, for example 0.4 to 1.5 g/100 ml was sufficient to further increase the viscosity to levels observed for Nutrilon A.R.1.

Combinations of pectin, XG and waxy starch showed good viscosities, but required an amount of pectin (HMA or HM) that was very close to the maximum amount allowed by regulation, and in addition higher concentrations of XG (0.08 to 0.1 g/100 ml) were needed. Some of the combinations tested showed large “fish eyes”. Also in some of the combination of pectin, XG and LBG tested, large “fish eyes” were observed.

From these experiments it became clear that the combination of XG/LBG with or without waxy starch was most promising in view of viscosity in the bottle and other product technological properties.

### Example 3: Viscosity Under Stomach Conditions. Selection of Optimum XG/LBG Thickeners Composition

Several combinations of XG and LBG were tested with the aim to identify the optimal XG/LBG thickeners concentrations needed to bring the kinetics of the stomach viscosity similar to the viscosity of the reference formula Nutrilon A.R.1.

First viscosity of Pepti Syneo thickened with various concentrations of LBG, waxy starch, XG or LBG-XG combinations was measured in the formula and during in vitro gastric digestion in time according to the method of example 1. As waxy starch did not contribute to the viscosity under stomach conditions (see example 1) it was not further tested in the combinations for the stomach conditions.

Subsequently, a matrix of XG/LBG concentrations was designed to cover XG and LBG concentrations to be tested. Xanthan gum was tested in several concentration, 0.02; 0.04; 0.05; 0.06 and 0.12 g/100 ml. Waxy starch: 2 g/100 ml. XG/LBG 0.02/0.48; 0.10/0.05; 0.035/0.02; 0.035/0.19; 0.034/0.35; 0.043/0.02; 0.043/0.19; 0.043/0.36; 0.043/0.043; 0.06/0.02; 0.06/0.19; 0.06/0.36; and 0.05/0.46 g/100 ml. A multiple linear regression model was developed to simulate gastric viscosity in time. Using this model, an equation solving program with multi factors found several optimums of XG and LBG to thicken the formulas under gastric conditions as close as possible to Nutrilon A.R.1. The prediction of gastric viscosity based on XG/LBG concentrations showed different behaviors in undigested/digested state when the ratio was close to 1/1 or deviating from 1/1. This finding was translated into 2 models, one for wherein the XG/LBG ratio was close to 1:1 and one for the situation where the ratio was deviating from 1/1. The optimal ratios were tested in the dynamic gastrointestinal model mimicking stomach digestion by an infant.

The two optimal concentrations as determined by the regression model were 1) XG/LBG in a wt/wt ratio of 1 to 1 and each fiber being present at 0.044/0.044 wt % in the reconstituted formula and 2) XG/LBG in a wt/wt ratio of 1 to 4.15 and LBG being present in a concentration of 0.235 wt % and XG being present in a concentration of 0.057 wt % in the reconstituted formula.

TABLE 2
Viscosities at 37° C. and at 10 s⁻¹ shear rate of different infant formulas mimicking gastric digestion

| | Formula | | | | | | | | | | | |
|---|---|---|---|---|---|---|---|---|---|---|---|---|
| | eHF | | A.R. | | eHF | | eHF | | eHF | | eHF | |
| | formula | | formula | | formula | | formula | | formula | | formula | |
| | Thickener g/100 ml | | | | | | | | | | | |
| | | | | | | | | | LBG 0.044 | | LBG 0.235 | |
| | — | | LBG 0.5 | | LBG 0.48 | | XG 0.05 | | XG 0.044 | | XG 0.057 | |
| Time [min] | Viscosity [mPa · s] (s.d) | | | | | | | | | | | |
| 0 | 13.7 | (11.4) | 78.8 | (9.7) | 73.1 | (14.8) | 44.3 | (12.0) | 57.3 | (5.3) | 77.5 | (13.7) |
| 10 | 13.6 | (8) | 18.8 | (5.6) | 18.9 | (2.9) | 26.7 | (5.2) | 42.7 | (2.1) | 33.2 | (5.3) |
| 30 | 2.8 | (1.6) | 13.8 | (5.1) | 6.0 | (1.4) | 14.8 | (3.0) | 16.1 | (3.3) | 12.7 | (2.1) |
| 50 | 1.4 | (1.0) | 12.2 | (4.6) | 3.5 | (0.4) | 10.2 | (2.0) | 14.5 | (0.4) | 8.0 | (2.1) |
| 70 | 3.8 | (6.8) | 8.6 | (2.6) | 2.8 | (1.1) | 9.6 | (0.6) | 13.8 | (0.9) | 8.5 | (2.0) |
| 90 | 1.6 | (0.4) | 5.8 | (1.4) | 2.3 | (0.2) | 8.8 | (0.3) | 12.1 | (0.4) | 6.8 | (0.6) |
| 120 | 1.0 | (0.3) | 3.1 | (0.6) | 1.9 | (0.3) | 7.1 | (0.2) | 9.4 | (1.1) | 7.4 | (0.3) |

The innovative hypoallergenic formulas with XG/LBG of 0.044/0.044 and 0.057/0.235 showed rheological gastric behavior that best mimicked the one found with a standard A.R. formula with proven efficacy. The XG/LBG 0.044/0.044 having as advantage that the overall fiber content is low, and the XG/LBG 0.057/0.235 having as advantage that also in the bottle (t=0) the viscosity is more comparable to the standard A.R. formula.

### Example 4: Effect of Thickeners in an Amino Acid Based Formula

Next to the efficacy of the thickeners in an extensively hydrolysed formula, also the effect of the thickeners in an amino acid formula was tested. Thickeners were added to Neocate Syneo, and infant formula comprising free amino acid as protein source and marketed for infants with severe cow's milk protein allergy.

TABLE 3
Viscosities at 37° C. and at 10 s⁻¹ shear rate of
different infant formulas mimicking gastric digestion

| | Formula | | | | | | | |
|---|---|---|---|---|---|---|---|---|
| | Standard A.R. | | AA based | | AA based | | Standard | |
| | formula | | formula | | formula | | Neocate | |
| | Thickener g/100 ml | | | | | | | |
| | | | LBG 0.044 | | LBG 0.235 | | | |
| | LBG 0.5 | | XG 0.044 | | XG 0.057 | | — | |
| Time [min] | Viscosity [mPa · s] (s.d) | | | | | | | |
| 0 | 78.8 | (9.7) | 24.8 | (4.6) | 59.0 | (8.9) | 17.9 | (3.3) |
| 10 | 18.8 | (5.6) | 20.6 | (5.3) | 18.2 | (0.8) | 7.5 | (4.6) |
| 30 | 13.8 | (5/1) | 10.7 | (2.1) | 9.9 | (0.4) | 0.2 | (0.9) |
| 50 | 12.2 | (4.6) | 11.1 | (1.1) | 7.7 | (1.4) | 0 | (0.9) |
| 70 | 8.6 | (2.6) | 10.8 | (1.0) | 6.6 | (0.2) | 0.2 | (1.0) |
| 90 | 5.8 | (1.4) | 10.3 | (0.5) | 7.1 | (1.6) | 0.1 | (0.5) |
| 120 | 3.1 | (0.6) | 7.7 | (0.8) | 6.2 | (0.5) | 0.9 | (0.1) |

As can be seen in Table 3 also in an amino acid based formula the combination of thickeners worked well under stomach conditions and was comparable to Nutrilon A.R and much higher that the product without thickeners.

### Example 5: Fermentation of Thickeners and Non-Digestible Oligosaccharides scGOS/lcFOS

Fecal samples were collected from a formula fed infant (5.5 months of age) and from a breastfed infant (3 months of age). The infants were without gastrointestinal problems and did not use antibiotics in the last month. Fecal samples were pooled, homogenized, divided in smaller aliquots, and mixed with glycerol (10%) in an anaerobic cabinet. Subsequent aliquot storage was at −80° C.

The non-digestible oligosaccharides were added at a concentration of 100 mg dietary fiber (DP≥2) per 6 ml of feces suspension, see the conditions in the table 4. Source of GOS was Vivinal GOS (Friesland Campina), source of lcFOS was Raftilin HP (Orafti). Sources of xanthan gum and locust bean gum are the same as in examples 1 and 2.

TABLE 4
Conditions in the fecal slurry fermentation

| | Condition (of dietary fibers) | Ratio (wt/wt) | Label in tables |
|---|---|---|---|
| 1. | scGOS/lcFOS | 9/1 | scG/lcF 9:1 |
| 2. | Xanthan Gum | 1 | XG |
| 3. | Locus Bean Gum | 1 | LBG |
| 4. | scGOS/lcFOS/Locus Bean Gum/ | 69.75/7.75/19/3.5 | scG/lcF/LBG/XG |
| | Xanthan Gum | (0.6975/0.075/0.19/0.035) | |

For the experiment, the fecal pool was defrosted in a water bath for 20 minutes at 37° C. The fecal pool was put thereafter in the anaerobic cabinet. Feces was mixed with the fermentation medium as 1:5 in a falcon tube. Samples of this fecal suspension were taken at t=0 and 6 ml of this suspension was added to a sterile falcon tube together with the substrate of interest and mixed thoroughly. Next, 6 ml of the feces/substrate suspension was put in a dialysis tube and air was removed in the empty space. The dialysis tube was put in a 100 ml Scott bottle filled with 100 ml dialysis medium. The Scott bottles were closed and incubated at 37° C. Samples of the dialysis medium (dialysate) and fecal suspension (lumen) were taken at t=24 and t=48 hours for determination of SCFA, D- and L-lactate, and gas volume. Fermentation medium (Mc Bain and MacFarlane) contained buffered peptone water 3.0 g/l, Yeast Extract 2.5 g/l, Tryptone 3.0 g/l, L-Cysteine-HCl 0.4 g/l, Bile salts 0.05 g/l, K2HPO4.3H2O 2.6 g/l, NaHCO30.2 g/l, NaCl 4.5 g/l, MgSO4.7H2O 0.5 g/l, CaCl2. 2H2O 0.3 g/l, FeSO4.7H2O 0.005 g/l. Ingredients were added one by one in 800 ml water, pH was adjusted to 5.5±0.1 with K2HPO4 or NaHCO₃ and volume was filled up to 1 liter. Medium was sterilized for 15 minutes at 121° C. and put in the anaerobic cabinet at least 16 hours before use.

Dialysis medium contained K2HPO4.3H2O 2.6 g/l, NaHCO₃0.2 g/l, NaCl 4.5 g/l, MgSO4.7H2O 0.5 g/l, CaCl2. 2H2O 0.3 g/l, FeSO4.7H2O 0,005 g/l. pH was adjusted to 5.5±0.1 with K2HPO4 or NaHCO₃. Medium was not sterilized because of forming of sediment. The medium was put in the anaerobic cabinet at least 16 hours before use.

The pH was measured by immersing a 423 pH-electrode (Mettler Toledo, Columbus, OH, USA), connected to a Handy-lab PH meter (Schott Glas, Mainz, Germany), directly in a sample.

Gas volume was determined with a unit to measure pressure and volume. The bottles were shaken thoroughly before measuring.

The SCFA acetic, propionic, n-butyric, iso-butyric, n-valeric, and isovaleric acids were quantitatively determined using a Shimadzu-GC2025 gas chromatograph with a flame ionization detector. As mobile phase hydrogen was used. The levels of SCFA were determined using 2-ethylbutyric acid as an internal standard. From the peak area a calibration curve was constructed and the concentration in the samples was calculated.

Lactate was determined enzymatically using an L-lactic acid detection kit with D- and L-lactate dehydrogenase (Boehringer Mannheim, Mannheim, Germany). First, samples were centrifuged for 10 min at 13.000 rpm at 4° C., then the supernatant was heated for 10 min at 100° C. to inactivate all enzymes and then the samples were centrifuged for 10 minutes at 13.000 rpm.

### Results:

Levels of isobutyric acid, valeric acid, and isovaleric acid were below detection limit. Lactic acid, predominantly L-lactic acid, was formed at t=24 h in the mixtures comprising scG/lcF. Interestingly, and advantageously the amount of lactic acid in the mixture of scG/lcF/XG/LBG was much higher than could be expected based on the fermentation properties of the individual components. As lactic acid is an intermediate metabolite, which is subsequently fermented by other bacteria, the synergistic excess of lactic acid was observed only in the earliest time point sampled. However, this is indicative of an increased activity of lactic acid producing bacteria.

Butyric acid, and especially propionic acid, were high when xanthan gum or locust bean gum was the sole fiber. As a result the SCFA profile showed less % of acetic acid, and higher % of butyric acid and propionic acid. Interestingly, when a combination of scGOS/lcFOS/Xanthan gum/Locust bean gum was tested, the formation of butyric acid, and especially propionic acid was lower in amount and percentage than could be expected based on the fermentation properties of the individual components. Advantageously also the amount of gas formed was lower than could be expected.

These effects were present at t=24 and 48 h. Table 5 shows the SCFA production and gas production after 48 h, a duration representative for the colonic transit time in an infant.

TABLE 5
Formation of fermentation end products (in μmol/g fiber)
after 48 h fermentation by infant intestinal microbiota

| | | | | | scG/lcF/LBG/XG |
|---|---|---|---|---|---|
| | scG/lcF 9:1 | XG | LBG | scG/lcF/LBG/XG | theory |
| Acetate (μmol/g) | 3750 | 2800 | 2400 | 3000 | 3460 |
| Butyrate (μmol/g) | 40 | 77 | 50 | 40 | 43.2 |
| Propionate (μmol/g) | 72 | 466 | 688 | 72 | 202 |
| Total SCFA (μmol/g) | 3862 | 3343 | 3138 | 3112 | 3705.2 |
| Acetate (%) | 97.1 | 83.8 | 76.5 | 96.4 | 92.7 |
| Butyrate (%) | 1.0 | 2.3 | 1.6 | 1.3 | 1.2 |
| Propionate (%) | 1.9 | 13.9 | 21.9 | 2.3 | 4.8 |
| Gas (ml) | 4.8 | 6.7 | 7 | 4.6 | 5.3 |

The same experiment was repeated but with a mixture of scGOS/lcFOS/LBG/XG of 0.8208/0.0912/0.044/0.044 and 0.6372/0.0708/0.235/0.057, respectively, and compared with XG alone, LBG alone, or scGOS/lcFOS alone.

Again, for both mixes that contained 4 fibers, the amount of and relative amount of butyrate was lower than expected based on the results obtained with XG alone, LBG alone or scGOS/lcFOS alone. In addition, the amounts of acetate and L-lactate (data shown in table 6) were increased and much higher than expected based on the results obtained with XG alone, LBG alone or scGOS/lcFOS alone.

TABLE 6
L-lactate production at t = 24

| | | | | scG/lcF/LBG/XG | | scG/lcF/LBG/X | |
|---|---|---|---|---|---|---|---|
| | scG/lcF | | | 0.821.0.091/0.044/ | | 0.637/0.071/0.235/ | |
| | 9:1 | XG | LBG | 0.044 | theory | 0.057 | theory |
| L-lactate | 625 | 90 | 90 | 3585 | 578 | 1030 | 469 |
| (μmol/g) | | | | | | | |

This is indicative for the mixture of scG/lcF/XG/LBG shifting the microbiota activity, lactic acid and SCFA formation synergistically in a direction more similar as observed in exclusively breastfed infants. As it is known that in preweaning healthy breastfed infants the microbiota produces high levels of acetic acid and L-lactic acid and low levels of butyric acid and propionic acid, when compared with standard formula fed infants or when compared with infants that suffer from intestinal microbial dysbiosis, or when compared with allergic infants (Wopereis et al. Pediatr Allergy Immunol 2014:25:428-438). Thickening agents such as the fibers XG and LBG are regarded as less acidic and more propionigenic and butyrogenic than conventional prebiotics such as scGOS and lcFOS, but the presence of scGOS and lcFOS could unexpectedly overcome these effects.

### Example 6: Fermentation of Thickeners and Non-Digestible Oligosaccharides scFOS/lcFOS

A fermentation experiment with fecal samples was performed in a way as described in example 5. The non-digestible oligosaccharides were added at a concentration of 100 mg dietary fibre (DP≥2) per 6 ml of feces suspension. All conditions are in the table below. Source of scFOS was Raftilose P95 (Orafti), source of lcFOS was Raftilin HP (Orafti). Sources of xanthan gum and locust bean gum are the same as in examples 1 and 2.

TABLE 7
Conditions in the fecal slurry fermentation

| | Condition (of dietary fibers) | Ratio (wt/wt) | Label in table |
|---|---|---|---|
| 1. | scFOS/lcFOS | 9/1 | scF/lcF 9:1 |
| 2. | Xanthan Gum | 1 | XG |
| 3. | Locus Bean Gum | 1 | LBG |
| 4. | scFOS/lcFOS/Locus Bean Gum/ | 69.75/7.75/19/3.5 | scF/lcF/LBG/XG |
| | Xanthan Gum | (0.6975/0.075/0.19/0.035) | |

### Results:

Levels of isobutyric acid, valeric acid, and isovaleric acid were below detection limit. Lactic acid was formed only in small amounts. The level of total SCFA (sum of acetic acid, propionic acid and butryric acid) was highest in the mixtures that contained scFOS/lcFOS.

The level of butyric acid, but especially propionic acid, was high when xanthan gum or locust bean gum was the sole fiber. As a result the SCFA profile showed less % of acetic acid, and higher % of butyric acid and % of propionic acid. Interestingly, when a combination of scFOS/lcFOS/XG/LBG was used, the formation of butyric acid and propionic acid was suppressed, and much lower amounts and percentages were produced than could be expected based on the fermentation properties of the individual components. Interestingly and advantageously also the amount of gas formed was lower than could be expected. This was observed at t=24 and 48 h. Table 8 shows the effect at 48 h, a duration representative for the colonic transit time in an infant. The amount of gas produced was high for scF/lcF mixture and low for XG or LBG, but in the mixtures of scF/lcF/LBG/XG the amount of gas produced was lower than expected based on the components alone.

TABLE 8
Formation of fermentation end products (mean in μmol/g
fiber) after 48 h fermentation by infant intestinal microbiota

| | | | | | scF/lcF/LBG/XG |
|---|---|---|---|---|---|
| | scF/lcF 9:1 | XG | LBG | scF/lcF/LBG/XG | theory |
| Acetate (μmol/g) | 1875 | 1400 | 1200 | 1650 | 1730 |
| Butyrate (μmol/g) | 45.25 | 38.5 | 25 | 20 | 41 |
| Propionate (μmol/g) | 67 | 233 | 344 | 36 | 125 |
| Total SCFA | 1987.25 | 1671.5 | 1569 | 1706 | 1897 |
| Acetate (%) | 94.4 | 83.8 | 76.5 | 96.7 | 90.6 |
| Butyrate (%) | 2.3 | 2.3 | 1.6 | 1.2 | 2.15 |
| Propionate (%) | 3.4 | 13.9 | 21.9 | 2.1 | 7.27 |
| D-lactate | 90 | 90 | 90 | 90 | 27 |
| L-lactate | 90 | 90 | 90 | 90 | 27 |
| Total lactate | 180 | 180 | 180 | 180 | 54 |
| Gas | 12.5 | 6.7 | 7 | 9.6 | 11.25 |

This is indicative for the mixture of scF/lcF/XG/LBG shifting the microbiota activity and SCFA formation synergistically in a direction more similar as observed in exclusively breastfed infants.

The same experiment was repeated but with a mixture of scFOS/lcFOS/LBG/XG of 0.8208/0.0912/0.044/0.044 and 0.6372/0.0708/0.235/0.057 Again for both mixes that contained the mixture of thickeners and non-digestible oligosaccharides, the amount and relative amount of butyrate was lower than expected based on the results obtained with XG alone, LBG alone or scFOS/lcFOS alone. In addition the amounts of gas produced was lower than expected based on the results obtained with XG alone, LBG alone or scGFOS/lcFOS alone.

### Example 7: Anti-Regurgitation Formula with Extensively Hydrolysed Whey Protein and Thickeners Suitable for Allergic Infants

A packed, powdered infant formula that that after reconstitution with water according to instructions on the pack contains per 100 ml (13.46 g powder in end volume of 100 ml):
66 kcal
1.6 g protein (extensively hydrolysed whey protein)
7.1 g digestible carbohydrates (mainly lactose)
3.4 g fat (vegetable oils, fish oil)
0.4 g Prebiotics: scGOS/lcFOS in a 9:1 ratio (source Vivinal GOS and Raftiline HP)
0.057 g Xanthan Gum
0.235 g Locust Bean Gum
   Minerals vitamins trace elements and other micronutrients as according to directives.

### Example 8: Anti-Regurgitation Formula with Extensively Hydrolysed Whey Protein and Thickeners Suitable for Allergic Infants

A packed, powdered follow on formula that that after reconstitution with water according to instructions on the pack contains per 100 ml (14.44 g powder in end volume of 100 ml):
68 kcal
1.6 g protein (extensively hydrolysed whey protein
7.8 g digestible carbohydrates (mainly lactose) and including 1.0 g waxy corn starch.
3.2 g fat (vegetable oils, fish oil)
0.7 g Prebiotics: scGOS/lcFOS in a 9:1 ratio (source Vivinal GOS and Raftiline HP)
0.044 g Xanthan Gum
0.044 g Locust Bean Gum
   Minerals vitamins trace elements and other micronutrients st as according to directives.

### Example 9: Anti-Regurgitation Formula with Free Amino Acids and Thickeners Suitable for Allergic Infants

A packed, infant formula that that after reconstitution with water, according to instructions on the pack contains per 100 ml (14.7 g powder in end volume of 100 ml):
67 kcal
1.8 g protein equivalent (free amino acids) L-Alanine, L-Arginine, L-Aspartic acid, L-Cystine, L-Glutamine, Glycine, L-Histidine, L-Isoleucine, L-Lysine, L-Methionine, L-Phenylalanine, L-Proline, L-Serine, L-Threonine, L-Tryptophan, L-Tyrosine, L-Valine, L-Carnithine
7.3 g digestible carbohydrates (mainly dried glucose syrop) and including 1.0 g waxy corn starch.
3.4 g fat (vegetable oils)
0.7 g Prebiotics: scFOS/lcFOS in a 9:1 ratio (source RaftiloseP95, and Raftiline HP)
0.044 g Xanthan Gum
0.044 g Locust Bean Gum
   Minerals vitamins trace elements and other micronutrients as according to directives.

1. A nutritional composition comprising digestible carbohydrates, lipids, protein and thickener suitable for providing nutrition to an infant or young child suffering from cow's milk protein allergy, wherein the protein is or essentially consists of extensively hydrolysed protein and/or free amino acids and wherein the thickener comprises a combination of xanthan gum and locust bean gum, the nutritional composition being in the form of a ready to drink liquid or in the form of a powder that upon reconstitution with water is a ready to drink liquid, wherein the xanthan gum is present in an amount of 0.03 to 0.07 g/100 ml, preferably 0.03 to 0.06 g/100 ml, even more preferably 0.04 to 0.05 g/100 ml ready to drink nutritional composition and locust bean gum is present in an amount of 0.03 to 0.07 g/100 ml, more preferably 0.03 to 0.06 g/100 ml, even more preferably 0.04 to 0.05 g/100 ml ready to drink nutritional composition and the wt/wt ratio of xanthan gum to locust bean gum is from 0.8 to 1.2.

2. The nutritional composition according to claim 1, wherein the wt/wt ratio of xanthan gum to locust bean gum is from 0.9 to 1.1.

3. The nutritional composition according to claim 1 or 2, which further comprises waxy starch as an additional thickener, preferably in an amount of 0.4 to 1.5 g/100 ml ready to drink nutritional composition, more preferably 0.4 to 0.8 g/100 ml ready to drink nutritional composition.

4. The nutritional composition according to any one of claims 1-3, wherein the protein is or essentially consists of extensively hydrolysed whey protein.

5. The nutritional composition according to any one of claims 1-4, which is an infant formula or a follow-on formula.

6. The nutritional composition according to any one of claims 1-5, wherein the shear viscosity of the ready to drink liquid at 37° C. and a shear rate of 10 s⁻¹ is from 20 to 100 mPa·s, more preferably from 25 to 90 mPa·s, even more preferably from 30 to 80 mPa·s.

7. The nutritional composition according to any one of claims 1-6, further comprising non-digestible oligosaccharide selected from the group consisting of fructo-oligosaccharides, non-digestible dextrin, galacto-oligosaccharides, xylooligosaccharides, arabino-oligosaccharides, arabinogalacto-oligosaccharides, gluco-oligosaccharides, glucomanno-oligosaccharides, galactomanno-oligosaccharides, mannan-oligosaccharides, chito-oligosaccharides, uronic acid oligosaccharides, sialyloligosaccharides and fuco-oligosaccharides, and mixtures thereof, preferably fructo-oligosaccharides.

8. The nutritional composition according to claim 7, wherein the amount of non-digestible oligosaccharide is at least 0.4 g/100 ml ready to drink nutritional composition, preferably from 0.4 to 0.8 g/100 ml, more preferably from 0.6 to 0.8 g/100 ml ready to drink nutritional composition.

9. The nutritional composition according to claim 7 or 8, wherein the non-digestible oligosaccharide is a mixture of long-chain fructo-oligosaccharides and short-chain-fructo-oligosaccharides or is a mixture of long-chain fructo-oligosaccharides and short-chain galacto-oligosaccharides.

10. The nutritional composition according to any one of claims 1-9, further comprising Bifidobacterium, preferably B. breve.

11. The nutritional composition according to any one of claims 1-10, which is a powder, suitable to be reconstituted with water to a ready to drink formula.

12. The nutritional composition according to any one of the preceding claims, for use in the dietary management of an infant or young child that suffers from allergy, preferably that suffers from cow's milk protein allergy.

13. The nutritional composition according to any one of claims 1-12, for use in the treatment or prevention of regurgitation or gastro-esophageal reflux (GER) in an infant or young child that suffers from allergy, preferably in an infant or young child that suffers from cow's milk protein allergy.

14. Use of a combination of xanthan gum and locust bean gum as thickeners in a hypoallergenic nutritional composition that is suitable for an infant or young child that suffers from cow's milk protein allergy, wherein the hypoallergenic nutritional composition comprises digestible carbohydrates, lipids and protein, wherein the protein is or essentially consists of extensively hydrolysed protein and/or free amino acids, wherein in the nutritional composition the xanthan gum is present in an amount of 0.03 to 0.07 g/100 ml, preferably 0.03 to 0.06 g/100 ml, even more preferably 0.04 to 0.05 g/100 ml ready to drink nutritional composition and locust bean gum is present in an amount of 0.03 to 0.07 g/100 ml, more preferably 0.03 to 0.06 g/100 ml, even more preferably 0.04 to 0.05 g/100 ml ready to drink nutritional composition and the wt/wt ratio of xanthan gum to locust bean gum is from 0.8 to 1.2, more preferably 0.9 to 1.1.

15. A process for preparing a nutritional composition according to any one of the preceding claims, comprising
a. providing a liquid nutritional composition comprising extensively hydrolysed protein and/or free amino acids as protein source, digestible carbohydrates, and lipids and optionally non-digestible oligosaccharide,
b. drying the liquid obtained in step a) to powder, preferably by spray drying, and
c. dry blending xanthan gum and locust bean gum and optional waxy starch, with the powder obtained in step b.
